(19) United States
(12) Patent Application Publication
(10) Pub. No.: US 2004/0199204 A1
(43) Pub. Date: Oct. 7, 2004

(54) Multifunctional surgical instrument

(76) Inventors: James W. Voegele, Cincinnati, OH (US); Robert P. Gill, Mason, OH (US); Wayne L. Poll, New Albany, OH (US)

Correspondence Address: PHILIP S. JOHNSON, JOHNSON & JOHNSON, ONE JOHNSON & JOHNSON PLAZA, NEW BRUNSWICK, NJ, 08933-7003, US

(21) Appl. No.: 10/777,708

(22) Filed: Feb. 12, 2004

Related U.S. Application Data
(60) Provisional application No. 60/447,542, filed on Feb. 14, 2003.

(51) Int. Cl. A61B 17/28

(52) U.S. Cl. 606/205; 606/210

(57) ABSTRACT

Disclosed is a minimally invasive surgical instrument that may be used in hand-assisted laparoscopic surgeries. The device is multifunctional surgical instrument that may be mounted directly on a surgeon's fingertip and inserted through an incision to allow the surgeon to manipulate tissue during a surgical procedure. The surgical instrument may be used for blunt dissection and allows for finger actuation of two opposing jaws to enable the surgeon to grasp and dissect tissue.

### CROSS REFERENCE TO RELATED APPLICATIONS

The present application claims the benefit of United States Provisional patent application serial No. 60/447,542, filed on Feb. 14, 2003, the contents of which are hereby incorporated herein by reference.

The present application is also related to U.S. patent applications, attorney docket no. END-5015NP, serial no. [______ ] and END-5016NP, serial no. [______ ] filed concurrently herewith.

### FIELD OF THE INVENTION

The present invention relates in general to the performance of a variety of surgical steps or procedures during surgical operations and, more particularly, to methods and apparatus for utilizing surgical instruments as an integral part of such surgical procedures to expedite and facilitate the surgical procedure and to extend a surgeon's sense of “feel”.

### BACKGROUND OF THE INVENTION

Abdominal surgery typically involves an incision in the abdominal wall large enough to accommodate a surgeon's hands, multiple instruments, and illumination of the body cavity. While large incisions simplify access to the body cavity during a surgery, it also increases trauma, requires extended recovery time, and can result in unsightly scars. In response to these drawbacks, minimally invasive surgical methods have been developed.

In minimally invasive abdominal surgery, or laparoscopic surgery, several smaller incisions are made into the abdominal wall. One of the openings is used to inflate the abdominal cavity with gas, which lifts the abdominal wall away from underlying organs and provides space to perform the desired surgery. This process is referred to as insufflation of the body cavity. Additional openings can be used to accommodate cannulas or trocars for illuminating and viewing the cavity, as well as instruments involved in actually performing the surgery, e.g., instruments to manipulate, cut, or resect organs and tissue.

While minimally invasive surgical methods overcome certain drawbacks of traditional open surgical methods, there are still various disadvantages. In particular, there is limited tactile feedback from the manipulated tissue to the surgeon hands. In non-endoscopic surgery, a surgeon can easily verify the identification of structures or vessels within a conventional open surgery incision. In particular the surgeon normally uses the sense of feel to verify the nature of visually identified operational fields. Further, in endoscopic surgery, tissue that is to be removed from the body cavity must be removed in pieces that are small enough to fit through one of the incisions.

Recently, new surgical methods have been developed that combine the advantages of the traditional and minimally invasive methods. It is sometimes referred to as hand assisted laparoscopic surgery (“HALS”). In these new methods, small incisions are still used to inflate, illuminate, and view the body cavity, but in addition, an intermediate incision is made into the abdominal wall to accommodate the surgeon's hand. The intermediate incision must be properly retracted to provide a suitable- sized opening, and the perimeter of the opening is typically protected with a surgical drape to prevent bacterial infection. A sealing mechanism is also required to prevent the loss of insufflation gases while the surgeon's hand is either inserted into or removed from the body cavity though the retracted incision.

While the hand provides a great deal of flexibility and retains the surgeon's sense of feel, fingers in themselves have limits as to their usefulness. Fingers lack the delicacy to pick up fine tissue. Fingers require making larger divisions when dissecting tissue. Fingers are subject to injury when holding tissue while energy modalities, such as ultrasound or RF, are used to treat the surgical site. Traditional instruments intended for conventional surgery i.e. forceps and graspers are too large for the limited body cavity environment. Traditional instruments also present the problem of being brought into and out of the laparoscopic site causing time-delaying deflation and re-insufflations of the body cavity. Laparoscopic equivalent instruments are delivered through a body wall port and have limited access to tissue.

U.S. Pat. Nos. 5,42,227; 6,149,642; 6,149,642; 5,925,064 disclose various aspects of laparoscopic surgery and fingertip devices for surgeon use.

With the advance represented by HALS procedures there is a need for improved fingertip surgical instrumentation that can take advantage of the increased freedom created by having a hand inside the body cavity.

The present invention overcomes the disadvantages of the prior art and provides the surgeon with a cost effective, yet efficiently flexible surgical instrument.

### BRIEF SUMMARY OF THE INVENTION

This need is met by the methods and apparatus of the present invention wherein a handheld or fingertip surgical device is used within a surgical field. In general the surgical instrument comprises a first jaw and a second jaw in an opposing relationship; a first lever arm attached to the first jaw and a second lever arm connected to the second jaw, wherein the first lever arm is arranged to permit movement of the first jaw relative to the second jaw and the second lever arm is arranged to permit movement of the second jaw relative to the first jaw.

In one aspect the surgical instrument is useful in minimally invasive surgery where the access to the surgical site is provided by a hand port. The surgical instrument may be manipulated within the surgeon's hand or the instrument may be slidably attached to the surgeon's finger and work as an extension of the surgeon's fingertip.

In another embodiment, the surgeon's finger may be supported within the surgical instrument by a strap or a band.

In another aspect, the invention features a method of performing a minimally invasive surgical procedure by creating in the patient an incision sized to accept a hand; inserting a hand and surgical instrument into the surgical site to perform blunt dissection; and actuating one or two lever arms to cause the jaws to move and allow the surgeon to grasp or dissect tissue.

Other features and advantages of the invention will become apparent from the following detailed description and from the claims.

### BRIEF DESCRIPTION OF THE FIGURES

These and other features, aspects, and advantages of the invention will become more readily apparent with reference to the following detailed description of a presently preferred, but nonetheless illustrative, embodiment when read in conjunction with the accompanying drawings. The drawings referred to herein will be understood as not being drawn to scale, except if specifically noted, the emphasis instead being placed upon illustrating the principles of the invention. In the accompanying drawings:

FIG. 1a is a cut-away perspective view of an exemplary use of the present invention;

FIG. 1b is an elevation view of one embodiment of the invention attached to a surgeon's finger;

FIG. 2a is a perspective view of the invention with the jaw closed and illustrating the movement of the jaw members with respect to the lever arms;

FIG. 2b is a perspective view of the invention with the jaw elements closed;

FIG. 2c is an exploded cut-away view illustrating detailed construction of an embodiment of the invention;

FIG. 2d is a perspective view of the invention with the jaw elements open;

FIGS. 3a-c are partial views of the jaw members illustrating the multifunctional capabilities of the invention; and

FIGS. 4a-g illustrate multiple examples how a surgeon can interface with the invention within a surgical site.

### DETAILED DESCRIPTION OF THE INVENTION

Before explaining the present invention in detail, it should be noted that the invention is not limited in its application or use to the details of construction and arrangement of parts illustrated in the accompanying drawings and description. The illustrative embodiments of the invention may be implemented or incorporated in other embodiments, variations and modifications, and may be practiced or carried out in various ways. Furthermore, unless otherwise indicated, the terms and expressions employed herein have been chosen for the purpose of describing the illustrative embodiments of the present invention for the convenience of the reader and are not for the purpose of limiting the invention.

It is understood that any one or more of the following-described embodiments, expressions of embodiments, examples, methods, etc. can be combined with any one or more of the other following-described embodiments, expressions of embodiments, examples, methods, etc.

While the methods and apparatus of the present invention are generally applicable to the performance of these surgical procedures during any operation, they are particularly applicable to their performance during HALS procedures and, accordingly, will be described herein with reference to this invention.

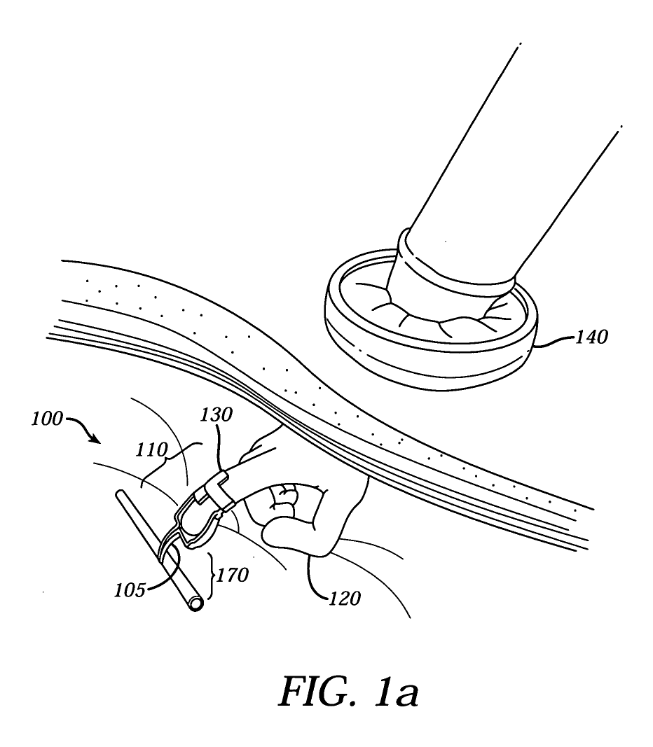

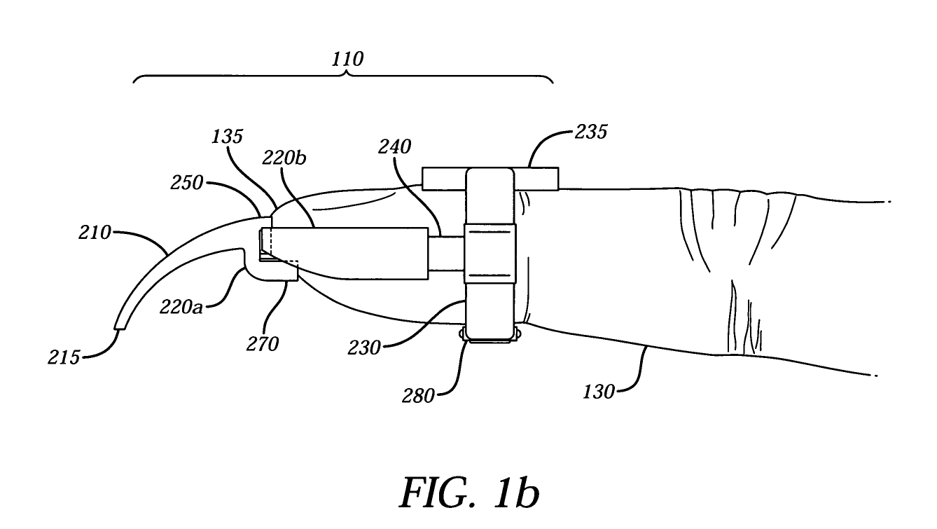

Referring now to FIG. 1 a, the environment for performing an endoscopic surgical procedure within an abdomen 100 is illustrated. A means for providing hand access, such as a lap disc 140, for example, model LD111 available from Ethicon Endo-Surgery, Cincinnati, Ohio, is placed into the abdominal wall. A surgeon places his arm and gloved hand 120 through the lap disc and into the abdomen cavity 100. In one embodiment of use, the index finger 130 (although any finger can be used) is capped with a finger device with a surgical instrument 110 having a working element 105. The working element 105 can be used to manipulate tissue, such as for example, a blood vessel 170 during a laparoscopic procedure.

FIG. 1b is a side view of a multifunctional fingertip surgical instrument 110 with a surgeon finger 130 inserted into the instrument 110 and resting against a tang 270. In addition to the tang 270, finger 130 is supported within instrument 110 via a band or a strap 230. Projecting from strap 235 is a spring element 240 that interfaces with a lever arm 220b. At the distal end of instrument 110 is a jaw assembly 210 for manipulating tissue. Strap 230 may be of any conventional design to allow the surgeon to slidably insert a working finger into instrument 110 and remove the finger with one-hand operation so the surgeon may easily change the interface with instrument 110 within the surgical site. Strap 230 includes an adjustable fastener 280 and optionally a support 235 that provides additional support between the surgeon's finger and instrument 110. Strap 230 may be configured in any of many conventional ways appropriate for surgical procedures and will not be discussed in detail.

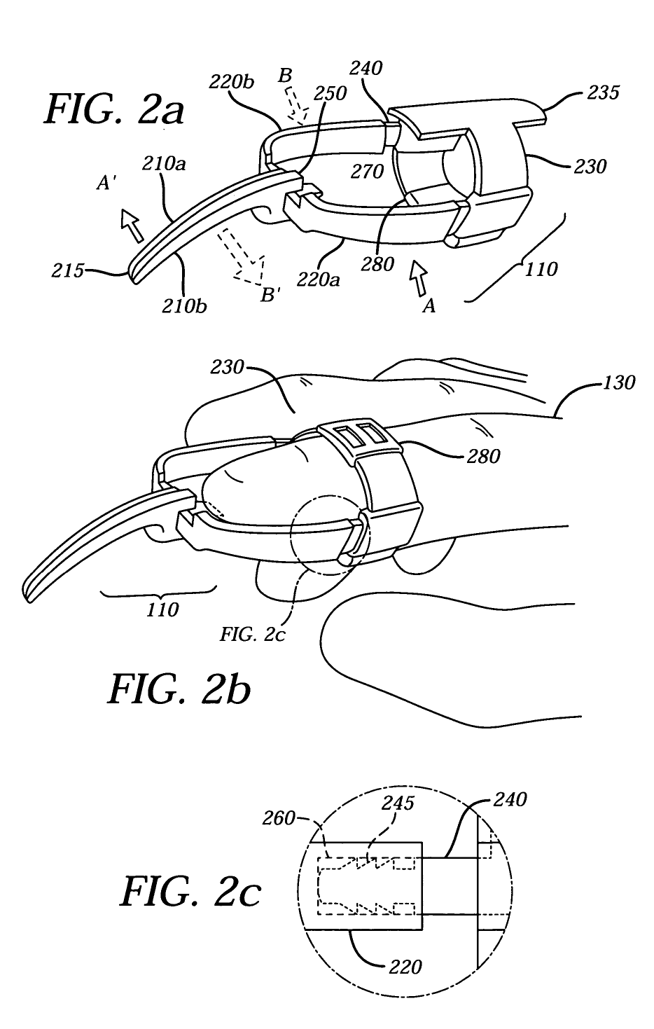

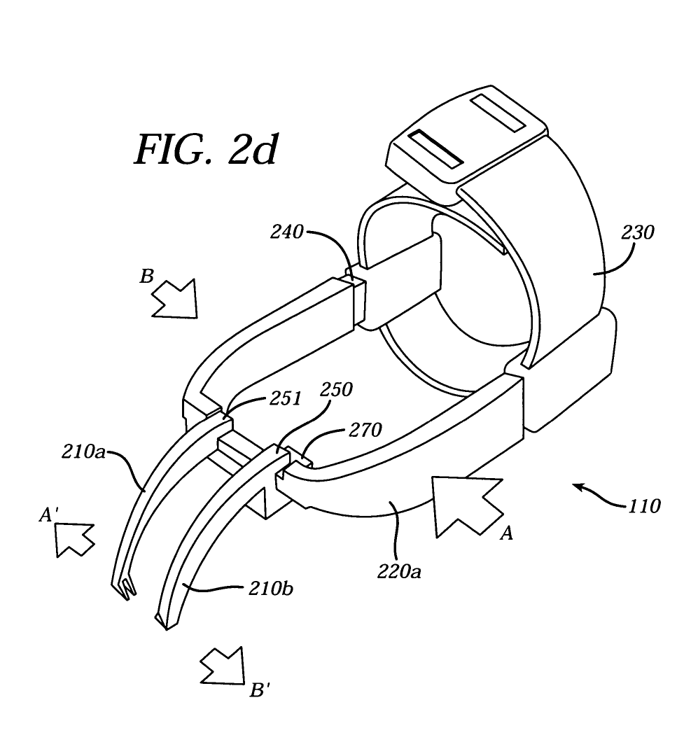

Referring now to FIGS. 2a-d, jaw 210 comprises opposing jaw members 210a and 210b and a jaw tip 215. Jaw member 210a is operatively connected to lever arm 220a and jaw member 210b is operatively connected to lever arm 220b as shown in FIG. 2d where shading differences are used to show lever arm connected to its corresponding jaw member. In this fashion, when the surgeon depresses lever arm 220a in the direction of A, then jaw member 210a will move in the A′ direction as noted and jaw member 210b will remain stationary. Likewise, when lever arm 220b is depressed in the B direction, then jaw member 210b will move in the B′ direction as noted and jaw member 210a will remain stationary. If the surgeon depresses both lever arms 220a and 220b simultaneously both jaw members 210a and 210b will move in their respective direction as illustrated in FIG. 2d. In this embodiment jaw member 210b includes a stop 250 for limiting the travel of jaw member 210b, and jaw member 210a includes a stop 251. Lever arms 220a, 220b attach to the ends of spring 240 that is made integral to band or strap 230. Spring 240 may attach to lever arms 220a and 220b by any conventional attachment method, for example a barb connector 245 interfacing with a slot 260 within the lever arms as shown in FIG. 2c.

Any surgical instrument material could be used to give form to the instrument 110. Metallic examples are: stainless steel or titanium. Plastic examples are: polycarbonate (pc) or polyetherimide (pei). The device construction may also be a composite of materials to create variations of hardness, clamping force, security of the ring attachment. In one embodiment jaws 210a-b and lever arms 220a-b are a molded urethane, spring 240 is a stainless steel spring and strap 235 is a flexible band molded of nylon. Additionally, barium sulfate may be added into one or more of the plastic components to provide a radio opaque presence to instrument 110 should it become necessary to find its location.

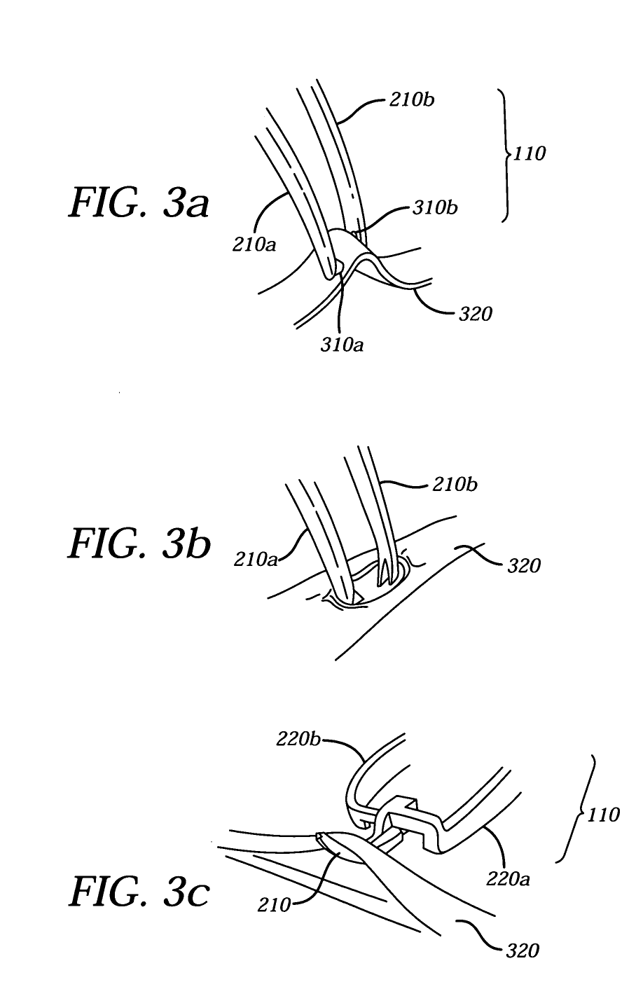

Referring now to FIGS. 3a-c instrument 100 may perform multiple functions during a surgical procedure. FIG. 3a demonstrates instrument 110 being used as a grasper. Jaws 210a, 210b have a distal mouse tooth male 310a and a mouse tooth female 310b, respectively, that mimic pick-up forceps for ease of collecting and holding tissue 320. FIG. 3b demonstrates instrument 110 being used as a dissector. Inserting the closed distal end of jaws 210a, 210b into tissue 320 and then depressing lever arms 220a and 220b causes jaws 210a, 210b to perform a desired blunt dissection. FIG. 3c demonstrates instrument 110 being used as a retractor/elevator. Curved Jaw 210 can be placed around tissue 320 or other structures for the purpose of retracting or elevating them. It is appreciated by one skilled in the art that jaw 210 may take on any variety of shape, such as straight, angled and have any number of surface finishes, such as smooth or serrated. In addition, jaw tip 215 may be blunted, sharp or other shape as required for a particular surgical function.

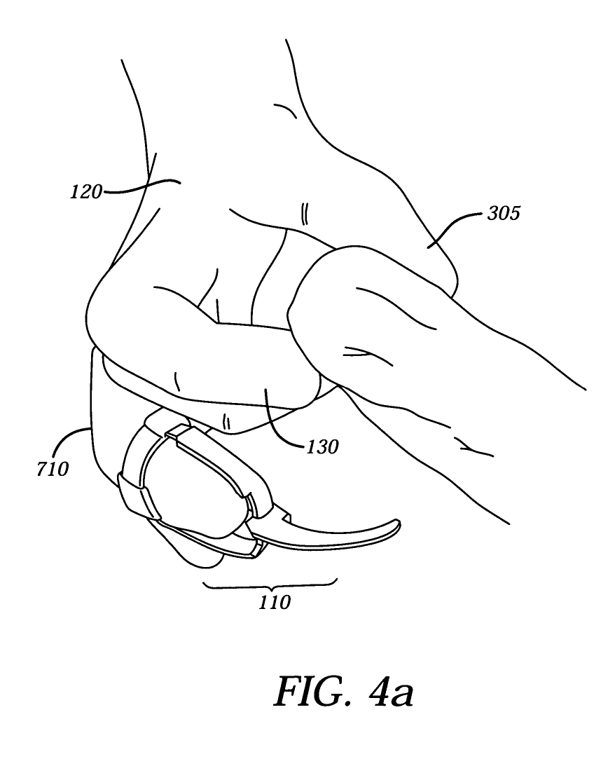

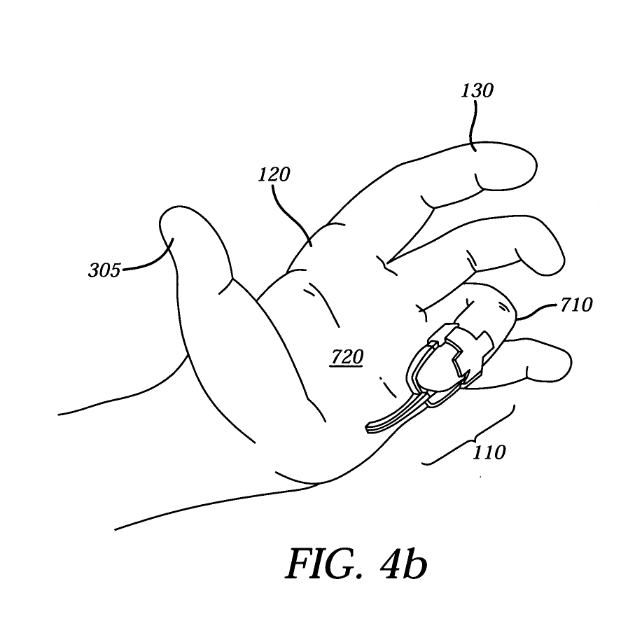

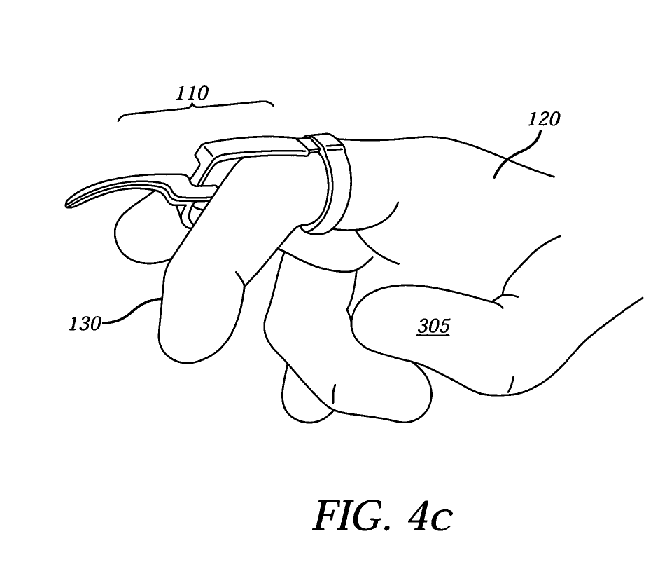

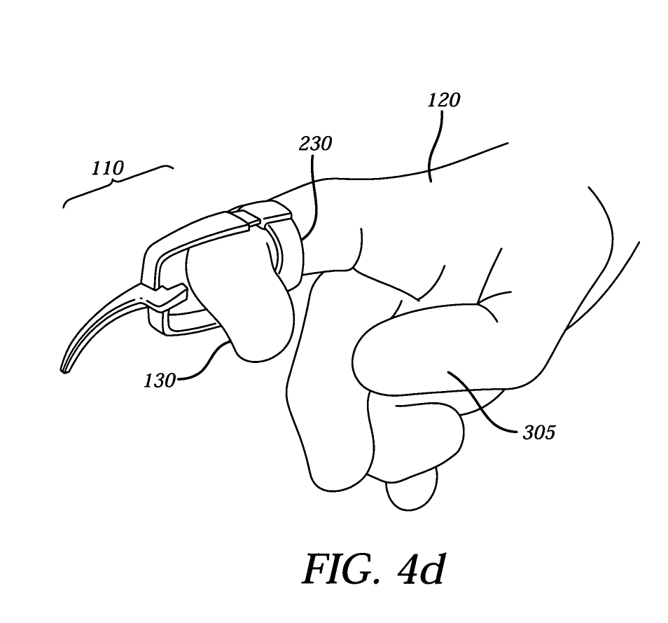

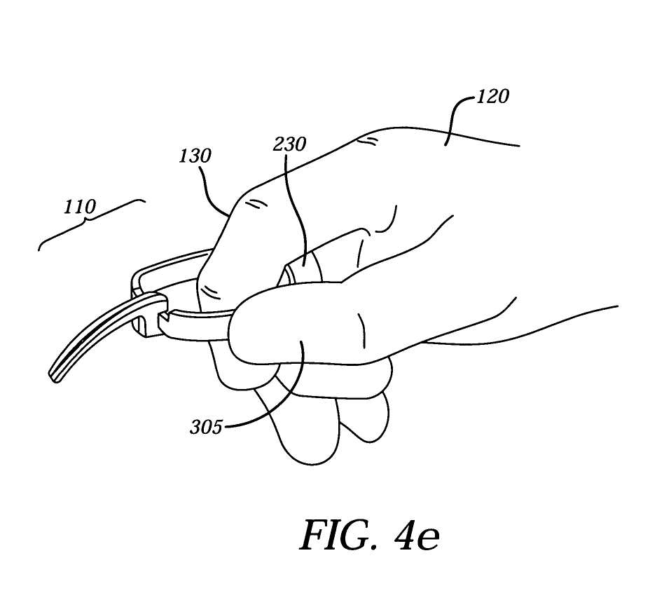

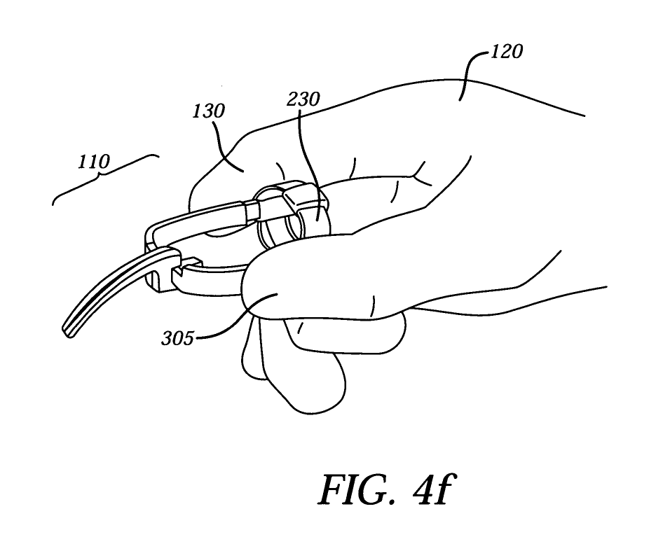

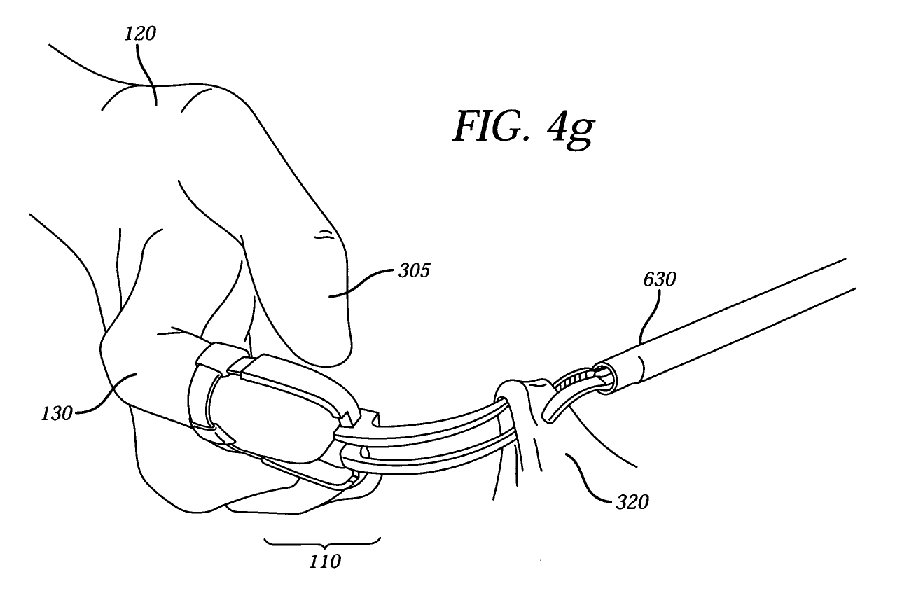

FIGS. 4a-g illustrate the utility of instrument 110 and how the simple design allows the surgeon to easily manipulate the instrument and remove the instrument from one finger and place on another. FIG. 4a demonstrates instrument 110 on a non-index finger 710 and useful in conjunction with manipulation of tissue between index finger 130 and thumb 305. FIG. 4b shows Instrument 110 tucked into the palm 720 of hand 120 for entry or exit to the surgical site via a lap disc or when performing functions with other fingers and it is desirable to have Instrument 110 out of the way. In FIG. 4c instrument 110 is in an off-use position yet still attached to index finger 130 to prevent it from being lost in the body cavity and/or readily accessible for its next use. Note that the surgeon has full use of fingers to ‘feel’ tissue within the body cavity as desired. FIG. 4d demonstrates the effectiveness of instrument 110 whereby index finger 130 hooks through band 230 for retraction purposes. Alternatively, as shown in FIG. 4e, the surgeon may grasp instrument 110 with index finger 130 and thumb 305 to manipulate tissue. In FIG. 4f the surgeon is able to use instrument 110 without it being attached to a finger, but functionally held by the surgeon. FIG. 4g illustrates instrument 110 being used to separate tissue 320 and holding the tissue in place while another surgical device 630 cuts the tissue. This device may apply energy from one of the well-known sources such as RF, ultrasound or laser. Note in this application the device provides a means of safety to the user by not placing their finger(s) in harms way.

While preferred embodiments of the present invention have been shown and described herein, it will be obvious to those skilled in the art that such embodiments are provided by way of example only. In addition, it should be understood that every structure described above has a function and such structure can be referred to as a means for performing that function. Numerous variations, changes, and substitutions will now occur to those skilled in the art without departing from the invention. Accordingly, it is intended that the invention be limited only by the spirit and scope of the appended claims.

What is claimed is:

1. A surgical instrument comprising:
a) a first jaw and a second jaw in an opposing relationship;
b) a first lever arm attached to the first jaw and a second lever arm connected to the second jaw, wherein the first lever arm is arranged to permit movement of the first jaw relative to the second jaw and the second lever arm is arranged to permit movement of the second jaw relative to the first jaw.

2. The surgical instrument of claim 1 further comprising a finger mount for slidably accepting a fingertip of a user.

3. The surgical instrument of claim 1 wherein the first and second jaws are curved.

4. A method of performing a minimally invasive surgical procedure in a patient comprising:
a) creating an incision to permit hand access within the patient;
b) introducing a hand instrument comprising:
i) a first jaw and a second jaw in an opposing relationship; and
ii) a first lever arm attached to the first jaw and a second lever arm connected to the second jaw,
c) actuating the first lever arm to move the first jaw, and
d) actuating the second jaw to move the second jaw relative to the first jaw.

5. The method of claim 4 further comprising the step of slidably engaging a finger with the hand instrument.

6. The method of claim 4 further comprising the step of actuating the first and second lever arms to grasp tissue.

7. The method of claim 4 further comprising the step of actuating the first and second lever arms to dissect tissue.

8. The method of claim 4 further comprising the step of performing blunt tissue dissection with at least one jaw.
